(19) United States
(12) Patent Application Publication
(10) Pub. No.: US 2004/0235045 A1
(43) Pub. Date: Nov. 25, 2004

(54) Method for disease prognosis based on Fc receptor genotyping

(75) Inventors: Harold Inge Nyland, Bergen (NO); Kjell-Morten Myhr, Bergen (NO); Christian Alexander Vedeler, Bergen (NO)

Correspondence Address: SUGHRUE MION, PLLC, 2100 Pennsylvania Avenue, NW, Washington, DC, 20037-3213, US

(73) Assignee: STIFTELSEN UNIVERSITETSFORSKNING BERGEN OF UNIFOB

(21) Appl. No.: 10/883,848

(22) Filed: Jul. 6, 2004

Related U.S. Application Data
(63) Continuation of application No. 09/599,002, filed on Jun. 22, 2000.
Application No. 09/599,002 is a continuation of application No. PCT/GB98/03872, filed on Dec. 22, 1998.

(30) Foreign Application Priority Data
Dec. 22, 1997 (GB) 9727055.7
Feb. 2, 1998 (GB) 9802207.2

(51) Int. Cl. C12Q 1/68

(52) U.S. Cl. 435/6

(57) ABSTRACT

A method of disease prognosis which involves determining the genotype of a human or non-human mammal subject for at least one Fc receptor, and identifying whether the determined genotype corresponds to a benign or non-benign prognosis for a disease selected from multiple sclerosis, myasthenia gravis, diabetes mellitus, cerebrovascular diseases, artherosclerosis, and Addison's disease.

This invention relates to a method of disease prognosis, in particular of multiple sclerosis, myasthenia gravis, diabetes mellitus, cerebrovascular and cardiocascular diseases, atherosclerosis, and Addison's disease.

Many diseases, in particular multiple sclerosis, diabetes and cardiovascular and cerebrovascular diseases have a widely different pattern of development over time with different patients. Thus some patients may have the disease but show minor or infrequent symptoms over many years while others, with apparently the same disease, may puffer relatively rapid deterioration leading even to total incapacity or death.

Where preventative, palliative or curative treatments for such diseases are known, however, these may, if administered unnecessarily, expose the patient to further risk (e.g. by suppressing the patients's immune response and so increasing the risk of other diseases), discomfort or expense.

As a result, the physician encountering a patient in the early stages of such a disease, or a patient found to have a genetic marker for susceptibility to such a disease, or a patient otherwise in an at-risk group for such a disease, faces a dilemma as to which course of curative, palliative or preventative treatment, if any, he should adopt.

There is therefore a need for a technique by means of which the progress of such diseases may be predicted for the individual patient so that, where the prognosis is good (i.e. disease progress is likely to be benign) unnecessary treatment may be avoided and where it is bad preventative action (e.g. immune modulating therapy for example immunization, diagnostic scanning, surgical intervention, etc.) may be taken for at-risk patients and therapeutic or palliative treatment may be given to early (and later) stage disease sufferers.

While there have been suggestions that there may be genetic markers for the progression of certain immune-related diseases, our investigations show that this does not appear in any way to be generally applicable (e.g. for poliomyelitis, chronic inflammatory demyelinating polyneuropathy, Guillain-Barre syndrome, rheumatoid arthritis, etc.). However we have now found that an individual's genotype for Fc receptors provides the basis for such prognostication for multiple sclerosis, myasthenia gravis, diabetes mellitus, cerebrovascular and cardiovascular diseases, atherosclerosis, and Addison's disease, a range of diseases which includes diseases which are not considered to be infection- or immune-related, e.g. in particular atherosclerosis and cardiovascular and cerebrovascular diseases.

Thus viewed from one aspect the invention provides a method of disease prognosis which involves determining the genotype of a human or non-human mammal subject for at least one Fc receptor, preferably an Fcγ receptor, and identifying whether the determined genotype corresponds to a benign or non-benign prognosis for a disease selected from multiple sclerosis, myasthenia gravis, diabetes mellitus, cerebrovascular and cardiovascular diseases, atherosclerosis, and Addison's disease.

By benign and non-benign prognoses, it is meant that the prognoses are more or less benign, e.g. good or not-so-good or bad or worse, etc.

This method may be considered to be one for determination of an indicator which may used by the physician in disease prognosis and, if necessary, the selection of appropriate treatments.

Viewed from a further aspect the invention provides a method of prophylaxis or therapy of a human or non-human mammal subject to combat a disease selected from multiple sclerosis, myasthenia gravis, diabetes mellitus, cerebrovascular and cardiovascular diseases, atherosclerosis, and Addison's disease, which method comprises determining the genotype of said subject for at least one Fc receptor, identifying whether the determined genotype corresponds to a benign or non-benign prognosis for said disease, and, where said determined genotype corresponds to a non-benign prognosis, carrying out a diagnostic imaging procedure on said subject, carrying out surgical intervention on said subject, or administering a prophylactically or therapeutically effective amount of a material prophylactically or therapeutically effective against said disease to said subject.

By way of example if the prognosis for atherosclerosis giving rise to heart or brain infarct is non-benign, early diagnostic imaging of the patient's vasculature may be recommendable and if stenoses are detected, surgical intervention, e.g. percutaneous transluminal angioplasty (PCTA), may reduce the likelihood of infarction so reducing future healthcare costs and improving the patient's future quality of life. Similarly, a non-benign prognosis according to the present invention, optionally coupled with detection of other risk factors such as high blood cholesterol, high homocysteine, high triglycerides, and high blood pressure may assist an individual to effect life style changes which will reduce the likelihood of development of atherosclerosis or of other cerebrovascular or cardiovascular disease, including the likelihood of infarction. Such changes may include cessation of smoking, change of diet, increase in regular exercise, reduction of stress, etc.

For diabetes mellitus, if the prognosis is non-benign, earlier insulin treatment, implantation of an insulin pump, or earlier pancreas or kidney transplant may prevent or delay onset of serious diabetes effects, e.g. diabetic retinopathy.

In the case of Type II (non-insulin dependant) diabetes patients, where the prognosis is non-benign, life style changes, weight loss, low-sugar diet and careful monitoring of blood sugar and/or insulin levels and possible early prescription of insulin may delay transition to or severity of Type I diabetes. For Type I patients, a non-benign diagnosis may support earlier insulin treatment, implantation of an insulin pump, etc. as mentioned above.

In the case of multiple sclerosis, a non-benign prognosis may predicate earlier prophylactic or therapeutic treatment, e.g. with interferons or gamma-globulin. Since such drugs are very expensive, the methods of the invention allow a more targetted use of medical and financial resources.

To determine the genotype of an individual for an Fc receptor, it is necessary to obtain a sample of the DNA of that individual. For this it is necessary to use FcR allele-specific binders (e.g. PCR primers or other materials capable of selectively binding to DNA or DNA fragments containing the particular FcR allele).

Accordingly, viewed from a further aspect, the invention provides the use of an FcR allele-specific binder for the manufacture of a composition for use in a method of prognosis, prophylaxis or therapy according to the invention.

Viewed from a further aspect the invention provides an FcR allele-specific binder for use in a method of prognosis, prophylaxis or therapy according to the invention.

Viewed from a still further aspect, the invention provides the use of a material prophylactically or therapeutically effective against a disease selected from multiple sclerosis, myasthenia gravis, diabetes mellitus, cerebrovascular and cardiovascular diseases, atherosclerosis, and Addison's disease for the manufacture of a medicament for use in the method of prophylaxis or therapy according to the invention.

Viewed from a still further aspect the invention provides the use of an Fc genotype in a method of prognosis of a disease selected from multiple sclerosis, myasthenia gravis, diabetes mellitus, cerebrovascular and cardiocascular diseases, atherosclerosis, and Addison's disease.

It should be stressed here that therapeutic treatment as referred to herein includes treatment to alleviate or reduce the occurrence of disease symptoms (i.e. palliative treatment) as well as curative treatment.

Viewed from a yet further aspect the invention provides a prognostic kit comprising at least one (preferably 2 or more, more preferably 4 or more, e.g. up to 12) FcR allele-specific binder and instructions for the performance of a method of prognosis, prophylaxis or therapy according to the invention.

The invention is particularly concerned with the genotypes for FcγR, i.e. for receptors for the Fc portion of immunoglobulin G (IgG). Such receptors occur on many cells, in particular leukocytes, microglia, endothelial cells, trophoblasts, keratinocytes and Schwann cells, e.g. monocytes, lymphocytes, granulocytes, neutrophils, and macrophages, and foam cells in atherosclerotic lesions (which are monocyte-derived cells).

Three main classes of human leukocyte FcγR have been identified, namely FcγRI (CD64), FcγRII (CD34) and FcγRIII (CD16). These show variability in their distribution on different cell types, in their strength of binding to IgG and their capability to bind to different IgG sub-classes. Within the FcγR classes, 8 genes and alternative splicing variants lead to a variety of receptor isoforms that have differences in structure and have distinct functional capacities. In addition to this variety, certain FcγR genes have allelic variants which affect their receptor function.

Thus for example FcγRIIA is expressed on monocytes, macrophages and neutrophils and has several allelic forms leading to FcγRIIA polymorphism. One variant contains histidine (131 H) while another contains arginine (131 R). The H/H variant has higher affinity for IgG2 than the R/R variant. Similarly, FcγRIIIB, which is only expressed on neutrophils, has several allelic forms with individuals homozygous for FcγRIIIB neutrophil antigen (NA)1 being more efficient in binding IgG1 and IgG3 than individuals homozygous for the NA2 allele. FcγRIIA and FcγRIIIB can also be simultaneously ligated leading to collaboration in the initiation of integrated cell functions.

The FcR genotype identified according to the invention is preferably FcγRIIIB and/or FcγRIIA, although more preferably both are identified. Nevertheless, the invention may be performed using other FcR genes which show allelic variation, especially FcR which are expressed on macrophage, neutrophil, microglia, endothelial cell or foam cell surfaces.

It must be emphasized here that the individual FcR genotype is not primarily being suggested as a marker for presence of or susceptibility to the selected disease, ie. whether or not the individual has a higher or lower than average likelihood of contracting the disease. Instead, identification of the FcR genotype according to the invention allows a prediction to be made of the severity and course of the disease should the individual contract it, or already have contracted it. Genetic markers (e.g. in the MHC region) for susceptibility to autoimmune and immune-related diseases are known, and in a further aspect the present invention provides a method of disease prognosis for a disease selected from multiple sclerosis, myasthenia gravis, diabetes mellitus, cerebrovascular and cardiovascular diseases, atherosclerosis, and Addison's disease, which method comprises determining the presence or absence of a genetic marker for susceptibility to said disease in the DNA of a human or non-human animal subject and determining the genotype of said subject for at least one Fc receptor, preferably an Fcγ receptor, and identifying whether the determined genotype corresponds to a benign or non-benign prognosis for said disease, said method optionally also involving carrying out a diagnostic imaging procedure on said subject, carrying out surgical intervention on said subject, or administering a prophylactally or therapeutically effective amount of a material prophylactally or therapeutically effective against said disease to said subject where said marker is present and said genotype corresponds to a non-benign prognosis. In further aspects, the invention provides prognostic kits and the use of FcR allele-specific binders and of therapeutic and prophylactic materials for the manufacture of compositions for use in such a method.

Viewed from a further aspect the invention provides a diagnostic assay for a disease selected from multiple sclerosis, myasthenia gravis, diabetes mellitus, cerebrovascular and cardiovascular diseases, atherosclerosis, and Addison's disease, said assay comprising obtaining a sample of DNA from a human or non-human mammal subject (e.g. involving separating such a sample deriving from a body fluid such as blood); and identifying the genotype of that DNA for a Fc receptor (preferably an FcγRIIA and an FcγRIIIB), for example by amplifying a segment of that DNA containing at least a characteristic part of the gene for that receptor and identifying the allele or alleles of the gene for that receptor present in that DNA; and optionally identifying the presence or absence in that DNA of a genetic marker for susceptibility to the selected disease, e.g. an MHC region marker for susceptibility to multiple sclerosis.

By a “diagnostic assay” is included a prognostic assay, ie. one which indicates not whether a disease condition is present but how it may progress.

Since the different FcR genotypes affect the binding characteristics of the receptor and thus for example the phagocytic activity of the cells carrying the receptors, and since the desirable binding and phagocytic activities may differ from disease to disease, it is relatively straightforward to determine the benign and non-benign genotypes for particular Fc receptors for the selected disease. This may be done by comparing the relative frequency of the different genotypes in a population of late-stage disease patients and thereby identifying which genotype or genotypes have significant occurrence in the sections of the population for which the disease progression has been benign or non-benign. This may for example mean comparing genotypes for patients with multiple sclerosis who can or cannot walk without support some years (e.g. at least 10 years) after disease onset, or comparing genotypes for patients with myasthenia gravis who have or have not developed thymomas, etc.

The FcR genotype of an individual may be determined from a sample of the individual's DNA (or a fragment thereof). Typically this may be obtained by taking a body fluid (e.g. blood, saliva or urine) or body tissue sample. Preferably the sample taken will be a blood sample.

Preferably, the DNA will be separated from other non-aqueous components of the sample, for example by cell lysis, solvent extraction and centrifugation.

The separated DNA may then be tested directly or may be amplified, e.g. using PCR with FcR allele specific primers, before determination. For direct testing, an allele-specific binder which carries or is conjugatable to a reporter (e.g. a radiolabel, a chromophore or an enzyme) should be used as in conventional direct or indirect binding assays. If DNA amplification is used, the amplified product may be separated on a gel. This is preferably done together with a standard DNA fragment produced by simultaneous amplification using a second primer effective for all subjects so as to avoid occurrence of false negatives for the particular FcR allele.

Many FcR genes have been identified in the literature and thus selection of appropriate allele-specific binder sequences is not problematic. Thus for FcγRIIA and FcγRIIIB for example the following PCR primers may be used:

FcνRIIA
EC2-131R:
5′CCAGAATGGAAAATCCCAGAAATTCTCTCG3′
EC2-131H:
5′CCAGAATGGAAAATCCCAGAAATTCTCTCA3′
TM1:
5′CCATTGGTGAAGAGCTGCCCATGCTGGGCA3′
Control 1:
5′GATTCAGTGACCCAGATGGAAGGG3′
Control 2:
5′AGCACAGAAGTACACCGCTGAGTC3′
FcνRIIIB
NA1:
5′CAGTGGTTTCACAATGTGAA3′
NA2:
5′CAATGGTACAGCGTGCTT3′
Reverse primer:
5′ATGGACTTCTAGCTGCAC3′
Control 1:
5′CAGTGCTTCCCAACCATTCCCTTA3′
Control 2:
5′ATCCACTCACGGATTTCTGTTGTGTTTC3′

Sequences such as these (e.g. the EC2-131R, EC2-131H, NA1 and NA2 sequences) or sequences with a high degree of homology therewith may be used as the allele-specific binders or as the binding domain of allele-specific binders in the kits of the invention.

For multiple sclerosis, FcγRIIIB NA1/NA1 and FcγRIIA H/H, together or separately are indicative of a benign prognosis. The order of increasing confidence of benign prognosis is: H/H; NA1/NA1; NA1/NA1+H/H.

For myasthenia gravis FcγRIIIB NA1/NA1 is indicative of a non-benign prognosis and R/R+NA2/NA2 is indicative of a benign prognosis.

For atherosclerosis and cardiovascular and cerebrovascular diseases, NA1/NA1 is indicative of a benign prognosis and NA2/NA2 of a non-benign prognosis.

For diabetes mellitus, H/H is indicative of a non-benign prognosis and R/R of a benign prognosis (e.g. lower likelihood of progression from Type II to Type I).

For Addison's disease, H/H is indicative of a non-benign prognosis, whereas R/R is indicative of a benign prognosis.

Where the prognosis according to the methods of the invention is #non-benign, the desired patient treatment may include: where the disease is or is not apparent, therapeutic (or prophylactic) treatment using the medicaments conventionally used for treatment of the particular disease (e.g. interferons or more preferably gamma-globulins for the treatment of multiple sclerosis); or a change of diet or cessation of smoking or alcohol consumption where the patient has, or has a susceptibility towards, diseases of the gut, kidneys, liver or cardiovascular or cerebrovascular system. In this regard, the medicaments used may be used in conventional dosage regimes.

The FcR genotyping according to the invention may be used not only to prognosticate disease progression but also to diagnose disease susceptibility for diabetes (especially Type I) and Addison's disease. Such diagnosis forms a further aspect of the invention. In such a method, presence of a “non-benign” genotype may be taken as an indicator of disease presence or susceptibility, e.g. to reinforce a diagnosis based on other tests, symptoms or indicators.

The invention will now be described further with reference to the following Examples and the accompanying drawings in which:

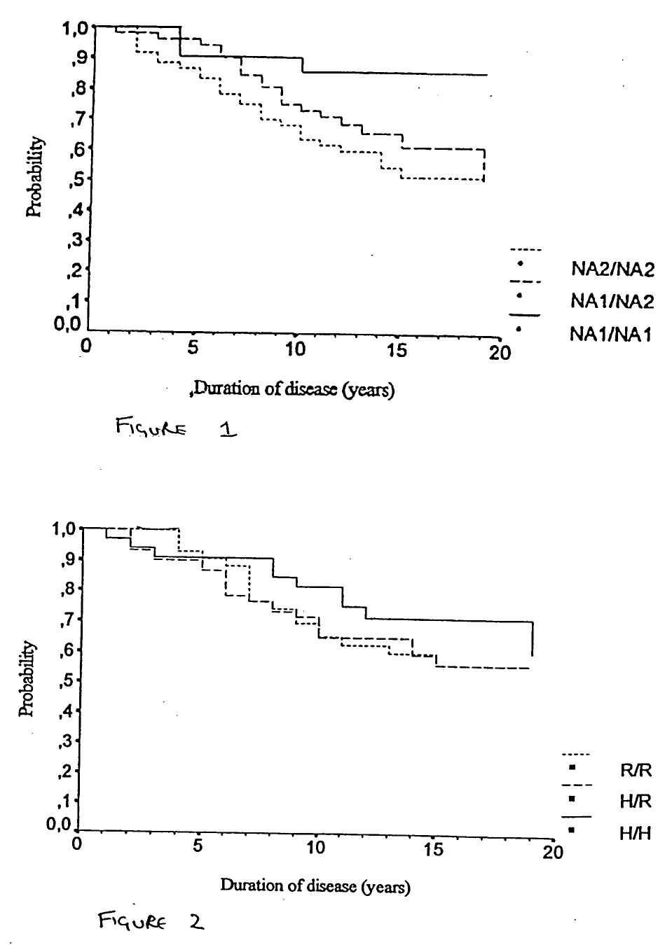

FIG. 1 is a plot of the probability of multiple sclerosis patients being able to walk without support (crutches or cane) correlated to duration of disease and FcγRIIIB genotype; and

FIG. 2 is a plot of the probability of multiple sclerosis patients being able to walk without support (crutches or cane) correlated to duration of disease and FcγRIIA genotype.

The following Examples set forth the results of studies of FcR genotype in multiple sclerosis and myasthenia gravis, atherosclerosis, stroke, diabetes mellitus and Addison's disease.

### EXAMPLE 1

Genotype Identification

Blood samples were taken from controls and patients suffering from multiple sclerosis or myasthenia gravis. DNA was extracted from whole blood with the QIAamp Blood kit (from Qiagen GmbH, Hilden, Germany) as described by the manufacturer. Thus frozen samples were thawed, and a cell-lysis buffer and QIAamp enzyme were added, and the samples were heated to 70° C. for 10 minutes. DNA was extracted using ethanol or isopropanol and the alcoholic sample were poured onto a DNA-binding column. The columns were rinsed with wash buffer, spun to dryness, and bound DNA was eluted with TRIS buffer, pH9. The DNA samples were collected in Eppendorf tubes and the DNA concentrations were measured. DNA fragments of at least about 30 kbp, concentration 25-50 ng/μL, were obtained. These could be stored frozen before PCR amplification. For PCR amplification, 50 to 100 ng DNA was used for each amplification with separate amplifications being performed for each allele for any given Fc receptor. Primers for FcγRIIA H, FcγRIIA R, FcγRIIIB NA1 and FcγRIIIB NA2 and for control DNA segments having the sequences set out above were used. These are obtainable from Medprobe and other PCR primer suppliers. PCR amplification was carried out on a Perkin Elmer automated PCR apparatus using an amplification refractory mutation PCR system comparable to that of Botto et al. (see Clin. Exp. Immunol. 104: 264-268 (1996)).

Two PCR reactions with two allele specific primers were carried out for each sample. Selective amplification of the allotypes was obtained by using primers of 30 nucleotides complementary to the sequence immediately adjacent to the polymorphism with the very 3′ nucleotide complementary to the crucial base. The EC2-131R primer contained guanine as 3′ base, whereas EC2-131H had adenine. The allele-specific primers contained a mismatch in position 3 from the 3′ end to further enhance the specificity in the annealing step of the PCR reaction. The antisense downstream primer (TM1) complementary to a sequence unique for the FcγRIIA gene in the Tm region did not discriminate between the two allotypes. The TM1 primer was used in both PCR reactions necessary for establishing the allotype. To verify that genomic DNA was present in the reactions, internal control primers amplifying a 270 bp from the TCR Vα22 gene were added. The PCR reactions were performed adding approximately 50 ng of genomic DNA into a 50 ml reaction containing 1×PCR buffer II (Perkin Elmer, N.J., USA), 0.0375 mM of each of the four dNTPs, 2.25 mM MgCl₂, 20 ng of each control primer, 100 ng of EC2-131R or EC2-131H primers in its respective reaction and 2.0 U of Taq DNA polymerase (Perkin Elmer). PCR conditions were: 94° C. for 3 minutes, followed by 45 cycles of 94° C. for 45 seconds, 63° C. for 30 seconds, 72° C. for 1 minute 30 seconds and a final extension step at 72° C. for 10 minutes. PCR products were identified on an about 1% agarose gel, visualised under UV light after 45 minutes at 70 volts. For control, PCR was performed on DNA from a patient known to be homozygous for the 131H allele and on the cell lines U937 (known to be homozygous for the 131R allele) and K562 (which is heterozygous).

The FcγRIII genotypes were determined using PCR with sequence-specific primers. Two PCR reactions with two allele specific primers were carried out for each sample. The NA1-specific primer was situated at position EC1 208-227 and had adenine at the 3′ end. To prevent mispriming and to enhance the specificity at position 4 from the 3′ end, adenine was replaced by thymine. The NA2-specific primer was situated at position EC1 130-147 and comprised two polymorphic sites. It had a T at the 3′ end and cytosine 7 nucleotides from the 3′ end. The reverse primer was situated at position EC1 331-348. Two human growth hormone primers (HGH-1 and HGH-2) were used as internal controls amplifying a 439 bp fragment of the HGH gene. The PCR reactions were performed adding approximately 50 ng of genomic DNA into a 40 μl reaction containing 1×PCR buffer (Perkin Elmer), 25 μM of each of the four dNTPs, 0.937 mM MgCl₂, 0.156 μM of each control primer, 0.625 μM of NA1 or NA2 primers in its respective reactions and 2.0 U of Taq DNA polymerase (Perkin Elmer). PCR conditions were: denaturation for 3 minutes at 94° C. followed by 33 cycles of 94° C. for 1 minutes, 57° C. for 2 minutes, 72° C. for 1 minute. A final extension step of 72° C. for 10 minutes was added. PCR products were identified on a 1% agarose gel, visualized under UV light after 45 minutes of 70 volts. For control, PCR was performed on DNA from patients with granulocytes expressing NA1 or NA2 determined by monoclonal antibodies.

### EXAMPLE 2

Population Studied and Results Obtained for Multiple Sclerosis (Ms) Patients

Population

136 Norwegian Caucasian MS patients (59 male, 77 female, aged 17 to 66, mean age 39.2 years) were studied. All had clinical onset of MS in the period 1976-1986 and had been diagnosed before 1st Jan. 1987. All patients were re-examined in 1995 with registering of disability according to the expanded disability status scale (EDSS) (see Kurtzke, Neurology, 33: 1444-145; (1983)). According to the diagnostic criteria of Poser et al. (see Ann Neurol. 13: 227-231 (1983)), 125 (91.9%) of the patients were classified (in 1995) as definite MS and 11 (8.1%) as probable MS. Mean duration of the disease was 14.9 years (range 9-19 years), and the initial course of the disease was relapsing-remitting (RRMS) in 109 (80.1%) and primary progressive (PPMS) in 27 (19.9%). Ninety-six, sex- and aged-matched Norwegian Caucasian healthy subjects from the same area served as controls.

Analysis

Chi-square tests were employed for analysis of categorial variables (genotypes and allele frequencies). EDSS showed an approximately normal distribution and parametric tests (one-way analysis of variance) were used in the analysis of disease progression (EDSS) related to genotypes. In addition, a multivariate regression analysis was performed with duration of disease, age-at-onset and sex as covariables to test for any residual effects of these variables. Life table survival analysis (Wilcoxon) was employed to test the probability for permanent need of walking assistance during the observation period.

Results

No significant differences between the allele frequencies were observed between the MS patients and the control group except that there was an abnormally low occurrence of FcγRIIIB NA1/NA1 in the primary progressive MS (PPMS) group (see Table 1 below).

TABLE 1
Distribution of FcΥRIIA and FcΥRIIIB genotypes in
patients with multiple sclerosis (MS) and controls

| | MS total | RRMS | PPMS | Controls |
|---|---|---|---|---|
| Genotype | n(%) | n(%) | n(%) | n(%) |
| FcΥRIIA | | | | |
| H/H | 33(24.3) | 27(24.8) | 6(22.2) | 18(18.8) |
| H/R | 60(44.1) | 48(44.0) | 12(44.4) | 45(46.9) |
| R/R | 43(31.6) | 34(31.2) | 9(33.3) | 33(34.4) |
| FcΥRIIIB | | | | |
| NA1/NA1 | 21(15.8) | 19(17.6) | 2(8.0) | 11(12.6) |
| NA1/NA2 | 52(39.1) | 45(41.7) | 7(28.0) | 41(47.1) |
| NA2/NA2 | 60(45.1) | 44(40.7) | 16(64.0) | 35(40.2) |

The correlation between FcγRIIIB genotype and benign as opposed to non-benign progression of MS is shown in FIG. 1 of the accompanying drawings. As may be seen, the NA1/NA1 genotype is significantly associated with the more benign prognosis. The correlation between FcγRIIA genotype and benign or non-benign progression of MS is shown in FIG. 2 of the accompanying drawings. As may be seen, the value of this genotype, on its own, as a prognostic indicator is lower than is that of the FcγRIIIB genotype.

Nine MS patients were homozygous for both FcγRIIA H/H and FcγRIIIB NA1/NA1 and these patients showed a significantly lower mean EDSS (ie. more benign disease progression) than the remaining patients.

Individuals with the H/H+NA1/NA1 genotype showed a mean EDSS score of 2.33 and 0% were PPMS. Individuals with the NA1/NA1 genotype showed a mean EDSS score of 2.85 and 10% were PPMS. Individuals with the NA2/NA2 genotype showed a mean EDSS score of 5.06 and 27% were PPMS.

### EXAMPLE 3

Population Studied and Results Obtained for Myasthenia Gravis (MG) Patients

The study included 30 MG patients and 49 healthy blood donors, all Norwegian Caucasians. Seven patients had a thymoma (lymphoepithelioma), 13 were late-onset MG patients (onset of MG symptoms after 40 years) and 10 were young-onset MG patients. Four patients had autoimmune diseases in addition to MG; one had Sjögrens disease; one had diabetes mellitus; one had rheumatoid arthritis; and one had systemic lupus erythematosus. The patients were classified according to the severity of the disease (see Mygland et al. J. Autoimmunity 6: 507-518 (1993)).

Analysis

Chi-square test, Fisher's exact test and Students t-test using statistical package for social sciences (SPSS) were applied to compare groups statistically.

Results

The frequency of occurrence of the FcγRIIA and FcγRIIIB genotypes was substantially similar for MG patients and healthy controls except for a noticeably higher incidence of the FcγRIIA H allele in the MG patients and a noticeably higher incidence of the FcγRIIA H/H and FcγRIIIB NA1/NA1 genotype in the MG patients with thymomas. The genotypes and allele frequencies are set out in Tables 2 and 3 below.

TABLE 2
Number of MG patients and controls with the various
FcγRIIA genotypes and allele frequencies

| | | | | Allele | |
|---|---|---|---|---|---|
| | Genotype | | | frequency | |
| | 131 R/R | 131 R/H | 131 H/H | 131R | 131H |
| MG total (n = 30) | 7(23%) | 13(43%) | 10(33%) | 0.46 | 0.54 |
| MG thymoma (n = 7) | 1(14%) | 1(14%) | 5(71%) | 0.21 | 0.79 |
| MG late-onset n = 13) | 4(31%) | 7(54%) | 2(15%) | 0.58 | 0.42 |
| MG young-onset | 2(20%) | 5(50%) | 3(30%) | 0.45 | 0.55 |
| (n = 10) | | | | | |
| Controls (n = 49) | 22(45%) | 18(37%) | 9(18%) | 0.63 | 0.37 |

TABLE 3
Number of MG patients and controls with the various
FcγRIIIB genotypes and allele frequencies

| | | | | Allele | |
|---|---|---|---|---|---|
| | Genotype | | | frequency | |
| | NA1/NA1 | NA1/NA2 | NA2/NA2 | NA1 | NA2 |
| MG total (n = 30) | 4(13%) | 15(50%) | 11(37%) | 0.38 | 0.62 |
| MG thymoma (n = 7) | 2(29%) | 4(57%) | 1(14%) | 0.57 | 0.43 |
| MG late-onset n = 13) | 1(8%) | 7(54%) | 5(38%) | 0.35 | 0.65 |
| MG young-onset | 1(10%) | 4(40%) | 5(50%) | 0.30 | 0.70 |
| (n = 10) | | | | | |
| Controls (n = 49) | 4(8%) | 28(57%) | 17(35%) | 0.36 | 0.64 |

The 4 MG patients with the NA1/NA1 genotype had more severe MG than patients with the NA1/NA2 and the NA2/NA2 FcγRIIIB genotypes. Moreover, autoimmune diseases in addition to MG did not occur in patients with the FcγRIIA 131 H/H or FcγRIIIB NA1/NA1 genotypes.

### EXAMPLE 4

Population Studied and Results Obtained for Atherosclerosis Associated Stroke

The study included 63 patients who had a cerebral infarct or a transient ischemic attack (TIA) and who, using ultrasound and angiographic studies, were found to have severe atherosclerosis (ie. stenosis of >70%) of the carotoid or vertebral arteries. Samples were analysed as in the preceeding Examples and the results are set out in Table 4 below.

TABLE 4

| | | | Genotype | | | | | | Allele Frequency | | | |
|---|---|---|---|---|---|---|---|---|---|---|---|---|
| | | | H/H | H/R | R/R | NA1/NA1 | NA1/NA2 | NA2/NA2 | H | R | NA1 | NA2 |
| Athero- | Patients^{@} | (54/63) | 10 | 27 | 17(31.5%) | 5(7.9%) | 25(39.7%) | 33(52.4%) | 47(43.5%) | 61(56.5%) | 35(27.8%) | 91(72.2%) |
| scle- | | | (18.5%) | (50.0%) | | | | | | | | |
| rosis | | | | | | | | | | | | |
| Healthy | Controls⁺ | (96/87) | 18 | 45 | 33(34.4%) | 11(12.6%) | 41(47.1%) | 35(40.2%) | 81(42.2%) | 111(57.8%) | 63(36.2%) | 111(63.8%) |
| | | | (18.8%) | (46.9%) | | | | | | | | |
| Non- | Controls* | (51/54) | 10 | 22 | 19(37.3%) | 6(10.9%) | 27(49.1%) | 22(40.0%) | 42(41.2%) | 60(58.8%) | 39(35.5%) | 71(64.5%) |
| athero- | | | (19.6%) | (43.1%) | | | | | | | | |
| scle- | | | | | | | | | | | | |
| rosis | | | | | | | | | | | | |

*Stroke patients without atherosclerosis of carotid or vertebral arteries (ultrasound studies). 51 for H/R and 54 for NA1/NA2 analysis
⁺96 for H/R, 87 for NA1/NA2 analysis
^{@}54 for H/R, 63 for NA1/NA2 analysis

### EXAMPLE 5

Diabetes Mellitus Types I and II

The study included 40 patients with Type I (insulin dependent) and 10 patients with Type II diabetes mellitus. Samples were analysed as in the previous Examples. The results are set out in Table 5 below

TABLE 5

| | | Genotype | | | | | |
|---|---|---|---|---|---|---|---|
| | | H/H | H/R | R/R | NA1/NA1 | NA1/NA2 | NA2/NA2 |
| Type I* | (38/40) | 17(44.7%) | 14(36.8%) | 7(18.4%) | 3(7.5%) | 21(82.5%) | 16(40.0%) |
| Controls⁺ | (96/87) | 18(18.8%) | 45(46.9%) | 33(34.4%) | 11(12.6%) | 41(47.1%) | 35(40.2%) |
| Type II | (10) | 1(10%) | 5(50%) | 4(40%) | 1(11.2%) | 4(44.4%) | 4(44.4%) |

*39 for H/R and 40 for NA1/NA2 analysis
⁺96 for H/R and 87 for NA1/NA2 analysis

The H/H genotype and the H allele occur with significantly greater frequency and the NA1/NA1 genotype with noticeably lower frequency for Type I patients.

### EXAMPLE 6

Addison's Disease

Addison's disease is a rare disease causing progressive destruction of the adrenal glands.

30 patients were studied and samples were analysed as in the previous Examples. The results are set out in Table 6 below.

TABLE 6

| | | Genotype | | | | | |
|---|---|---|---|---|---|---|---|
| | | H/H | H/R | R/R | NA1/NA1 | NA1/NA2 | NA2/NA2 |
| Type I* | (30/27) | 9(30.0%) | 21(70.0%) | 0(0%) | 4(14.8%) | 12(44.4%) | 11(40.7%) |
| Controls⁺ | (96/87) | 18(18.8%) | 45(46.9%) | 33(34.4%) | 11(12.6%) | 41(47.1%) | 35(40.2%) |

*30 for H/R and 27 for NA1/NA2 were analysed
⁺96 for H/R and 87 for NA1/NA2 were analysed The H/H genotype and the H allele correlates significantly.

1-14. (Cancelled).

15. A method comprising the steps of:
(a) determining the genotype of DNA encoding at least one Fc receptor, wherein said DNA is obtained from a test mammalian subject; and
(b) comparing the thus determined genotype to the genotype of DNA encoding a corresponding Fc receptor obtained from a normal mammalian subject or the genotype of DNA encoding a corresponding Fc receptor obtained from a diseased mammalian subject, wherein said diseased mammalian subject is a mammalian subject afflicted with a disease selected from the group consisting of multiple sclerosis, myasthenia gravis, diabetes mellitus, non-immune related cerebrovascular disease, non-immune related cardiovascular disease, atherosclerosis and Addison's disease,
wherein when the determined genotype for the DNA obtained from the test mammalian subject corresponds to the genotype of DNA obtained from said normal mammalian subject, a benign prognosis is made for the test mammalian subject; and
wherein when the determined genotype of DNA obtained from the test mammalian subject corresponds to the genotype of DNA obtained from said diseased mammalian subject, a non-benign prognosis is made for the test mammalian subject.

16. The method of claim 15, wherein said Fc receptor is an Fcγ receptor.

17. The method of claim 16, wherein said Fcγ receptor is FcγRIIA, FcγRIIIB or a combination thereof.

18. The method of claim 15, wherein the genotype is FcγRIIA H or R, FcγRIIIB NA1 or NA2, or a combination thereof.

19. The method of claim 15, wherein when a non-benign prognosis is made, said method further comprises the step of:
(c) determining the presence or absence of a genetic marker for susceptibility to said disease in the test mammalian subject.

20. The method of claim 15, wherein when a non-benign prognosis is made, said method further comprises the step of:
(c) subjecting the test mammalian subject to diagnostic imaging.

21. The method of claim 15, wherein when a non-benign prognosis is made, said method further comprises the step of:
(c) subjecting the test mammalian subject to surgical intervention against said disease.

22. The method of claim 15, wherein when a non-benign prognosis is made, said method further comprises the step of:
(c) administering, to the test mammalian subject, a prophylactically or therapeutically effective amount of a prophylactic or therapeutic material against said disease.

23. The method of claim 19, wherein when a non-benign prognosis is made and the presence of said genetic marker for susceptibility to said disease is found in the test mammalian subject, said method further comprises the step of:
(d) administering, to the test mammalian subject, a prophylactically or therapeutically effective amount of a prophylactic or therapeutic material against said disease.

24. The method of claim 19, wherein said method further comprises the step of:
(d) subjecting the test mammalian subject to diagnostic imaging.

25. The method of claim 19, wherein said method further comprises the step of:
(d) subjecting the test mammalian subject to surgical intervention against said disease.

26. A diagnostic method comprising the steps of:
(a) obtaining test DNA from a test mammalian subject, wherein said test DNA encodes at least one Fc receptor;
(b) determining the genotype of thus obtained test DNA; and
(c) comparing the thus determined genotype to the genotype of DNA encoding a corresponding Fc receptor obtained from a normal mammalian subject and to the genotype of DNA encoding a corresponding receptor obtained from a diseased mammalian subject, wherein said diseased mammalian subject is a mammalian subject afflicted with a disease selected from the group consisting of multiple sclerosis, myasthenia gravis, diabetes mellitus, non-immune related cerebrovascular disease, non-immune related cardiovascular disease, atherosclerosis and Addison's disease,
wherein when the determined genotype of the test DNA corresponds to the genotype of DNA obtained from said diseased mammalian subject, said test mammalian subject is diagnosed with said disease.

27. The method of claim 26, wherein said method further comprises the step of:
(d) determining the presence or absence of a genetic marker for susceptibility to said disease in the test mammalian subject.

28. The method of claim 15, wherein said genotype is determined using an Fc receptor allele-specific binder.


SEQUENCE LISTING (10 sequences)

SEQ ID NO: 1 - DNA, 30 bp, Human
ccagaatggaaaatcccagaaattctctcg

SEQ ID NO: 2 - DNA, 30 bp, Human
ccagaatggaaaatcccagaaattctctca

SEQ ID NO: 3 - DNA, 30 bp, Human
ccattggtgaagagctgcccatgctgggca

SEQ ID NO: 4 - DNA, 24 bp, Human
gattcagtgacccagatggaaggg

SEQ ID NO: 5 - DNA, 24 bp, Human
agcacagaagtacaccgctgagtc

SEQ ID NO: 6 - DNA, 20 bp, Human
cagtggtttcacaatgtgaa

SEQ ID NO: 7 - DNA, 18 bp, Human
caatggtacagcgtgctt

SEQ ID NO: 8 - DNA, 18 bp, Human
atggacttctagctgcac

SEQ ID NO: 9 - DNA, 24 bp, Human
cagtgcttcccaaccattccctta

SEQ ID NO: 10 - DNA, 28 bp, Human
atccactcacggatttctgttgtgtttc